(19) United States
(12) Patent Application Publication
(10) Pub. No.: US 2020/0237779 A1
(43) Pub. Date: Jul. 30, 2020

(54) COMBINATION THERAPY WITH A BET INHIBITOR AND A BCL-2 INHIBITOR

(71) Applicant: Hoffmann-La Roche Inc., Little Falls, NJ (US)

(72) Inventors: Mark D. DEMARIO, New York, NY (US); Thomas FRIESS, Penzberg (DE); Astrid Alexandra RUEFLI-BRASSE, Basel (CH)

(73) Assignee: Hoffmann-La Roche Inc., Little Falls, NJ (US)

(21) Appl. No.: 16/752,570

(22) Filed: Jan. 24, 2020

Related U.S. Application Data
(63) Continuation of application No. PCT/EP2018/070006, filed on Jul. 24, 2018.
(60) Provisional application No. 62/537,159, filed on Jul. 26, 2017.

(51) Int. Cl. A61K 31/5517; A61K 31/496; A61P 35/00

(52) U.S. Cl. CPC A61K 31/5517; A61K 45/06; A61P 35/00; A61K 31/496

(57) ABSTRACT

The present invention is directed to the combination therapy of multiple myeloma with a BET inhibitor and a Bcl-2 inhibitor.

### CROSS-REFERENCE TO RELATED APPLICATION

This application is a continuation of International Patent Application No. PCT/EP2018/070006, having an international filing date of Jul. 24, 2018, the entire contents of which are incorporated herein by reference in its entirety, which claims benefit to U.S. Patent Application No. 62/537,159 filed Jul. 26, 2017.

### FIELD OF INVENTION

The present invention is directed to the combination therapy, in particular of multiple myeloma with a BET inhibitor and a Bcl-2 inhibitor.

Multiple myeloma (MM) is a debilitating malignancy that is part of a spectrum of diseases ranging from monoclonal gammopathy of unknown significance (MGUS) to plasma cell leukemia. First described in 1848, MM is characterized by a proliferation of malignant plasma cells and a subsequent overabundance of monoclonal paraprotein (M protein).

The presentation of MM can range from asymptomatic to severely symptomatic, with complications requiring emergent treatment. Systemic ailments include bleeding, infection, and renal failure; pathologic fractures and spinal cord compression may occur.

Epigenetic dysregulation plays an important role in driving the aberrant gene expression patterns seen in a variety of hematologic malignancies. As many epigenetic alterations are reversible, these factors have drawn considerable attention as potential antineoplastic targets. One particular target of significant clinical interest is the bromodomain and extra-terminal (BET) family of proteins, which includes BRD2, BRD3, BRD4, and the testis-specific BRDT. Bromodomains (BRDs) are protein domains that possess a high affinity for binding to acetylation motifs, including acetylated histone proteins within chromatin. The BET family of proteins binds to acetylated chromatin and regulates gene transcription.

Selective inhibition of the interaction between BET proteins and acetylated chromatin has resulted in significant activity in preclinical models of acute leukemia, lymphoma, and multiple myeloma (MM). Targeting BET proteins could specifically target transcription of oncogenes and genes critical to disease development and progression.

Bcl-2 proteins play a role in many diseases, particularly in cancer, leukemia, immune and autoimmune diseases. Bcl-2 proteins are said to be involved in bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer. Overexpression of Bcl-2 proteins correlate with resistance to chemotherapy, clinical outcome, disease progression, overall prognosis or a combination thereof in various cancers and disorders of the immune system.

It was surprisingly found that the combination of a BET inhibitor with a Bcl-2 inhibitor showed significantly enhanced efficacy against multiple myeloma, causing a distinct tumor regression. Surprisingly, the tumor regression with this combination is more than additive, i.e.. superior to the cumulated anti-tumor efficacy induced by each of the two components separately.

The invention thus relates in particular to:

A BET inhibitor and a Bcl-2 inhibitor for use as a medicament;

A BET inhibitor and a Bcl-2 inhibitor for use in the treatment of multiple myeloma;

The BET inhibitor and Bcl-2 inhibitor for use according to the invention, wherein the BET inhibitor is 2-[(S)-4-(4-Chloro-phenyl)-2,3,9-trimethyl-6H-1-thia-5,7,8,9a-tetraaza-cyclopenta[e]azulen-6-yl]-N-[3-(4-methyl-piperazin-1- yl)-propyl]-acetamide (RG6146), INCB-054329, INCB-057643, GSK525762, GS-5829, CPI-0610, Birabresib, PLX51107, ABBV-075, BI 894999, FT-1101, ZEN-3694, GSK-2820151 or BMS-986158;

The BET inhibitor and Bcl-2 inhibitor for use according to the invention, wherein the BET inhibitor is 2-[(S)-4-(4-Chloro-phenyl)-2,3,9-trimethyl-6H-1-thia-5,7,8,9a-tetraaza-cyclopenta[e]azulen-6-yl]-N-[3-(4-methyl-piperazin-1- yl)-propyl]-acetamide (RG6146);

The BET inhibitor and Bcl-2 inhibitor for use according to the invention, wherein the Bcl-2 inhibitor is venetoclax, navitoclax, obatoclax, S-055746 or PNT-2258;

The BET inhibitor and Bcl-2 inhibitor for use according to the invention, wherein the Bcl-2 inhibitor is venetoclax;

The BET inhibitor and a Bcl-2 inhibitor for use according to the invention, comprising one or more additional other cytotoxic, chemotherapeutic or anti-cancer agents;

The BET inhibitor and a Bcl-2 inhibitor for use according to the invention, comprising ionizing radiation enhancing the effects of said agents;

A pharmaceutical composition comprising a BET inhibitor and a Bcl-2 inhibitor and one or more pharmaceutically acceptable excipients;

A pharmaceutical composition comprising a BET inhibitor and a Bcl-2 inhibitor and one or more pharmaceutically acceptable salt thereof for use in the treatment of multiple myeloma;

The use of a BET inhibitor and a Bcl-2 inhibitor for the manufacture of a medicament for the treatment of multiple myeloma;

The use of a BET inhibitor and a Bcl-2 inhibitor in the treatment of multiple myeloma;

A method of treatment of multiple myeloma comprising the administration of a BET inhibitor and a Bcl-2 inhibitor to a patient in the need thereof;

A kit comprising a BET inhibitor and a Bcl-2 inhibitor for the simultaneous, separate or sequential administration of said BET inhibitor and Bcl-2 inhibitor;

A kit according to the invention for use in the treatment of multiple myeloma;

A pharmaceutical composition, a use, a method or a kit according to the invention, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl- piperazin-1-yl)-propyl]-acetamide (RG6146), INCB-054329, INCB-057643, GSK525762, GS-5829, CPI-0610, Birabresib, PLX51107, ABBV-075, BI 894999, FT-1101, ZEN-3694, GSK-2820151 or BMS-986158;

A pharmaceutical composition, a use, a method or a kit according to the invention, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl- piperazin-1-yl)-propyl]-acetamide (RG6146);

A pharmaceutical composition, a use, a method or a kit according to the invention, wherein the Bcl-2 inhibitor is venetoclax, navitoclax, obatoclax, S-055746 or PNT-2258; and

A pharmaceutical composition, a use, a method or a kit according to the invention, wherein the Bcl-2 inhibitor is venetoclax.

The BET inhibitor and Bcl-2 inhibitor according to the invention are thus administered in combination (or co-administered).

The invention thus relates to a BET inhibitor and a Bcl-2 inhibitor for use in combination according to the invention.

The invention thus relates to a BET inhibitor and a Bcl-2 inhibitor for use in combination as a medicament, in particular for use in combination in the treatment of multiple myeloma.

In one embodiment, the BET inhibitor is a compound selected from the compounds described in WO 2011/143669. Methods of producing said BET inhibitors are also disclosed in WO 2011/143669.

Most preferably, the BET inhibitor is 2-[(S)-4-(4-Chloro-phenyl)-2,3,9-trimethyl-6H-1-thia-5,7,8,9a-tetraaza-cyclopenta[e]azulen-6-yl]-N-[3-(4-methyl- piperazin-1-yl)-propyl]-acetamide as in the formula below, or a salt thereof. Example JQ35 of WO 2011/143669 describes a method for its preparation.

The preferred BET inhibitor is depicted in the following formula:

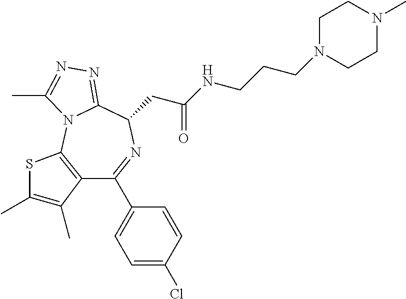

The above BET inhibitor is also known as RG6146, JQ35 or TEN-010.

In one embodiment, the Bcl-2 inhibitor is a compound selected from the compounds described in WO 2010/138588. Methods of producing said Bcl-2 inhibitors are also disclosed in WO 2010/138588.

Most preferably, the Bcl-2 inhibitor is 4-(4-{[2-(4-chlorophenyl)-4,4-dimethylcyclohex-1-en-1-yl]methyl}piprazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide as in the formula below or a salt thereof. Example 5 of WO 2010/138588 describes methods for preparation of said Bcl-2 inhibitor.

The preferred Bcl-2 inhibitor is depicted in the following formula:

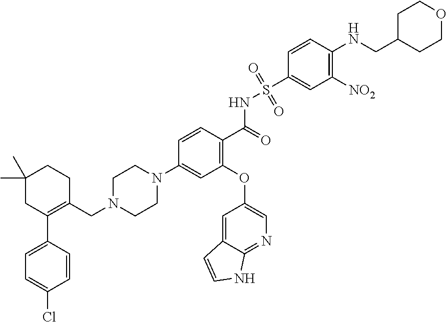 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

The above Bcl-2 inhibitor is also named ABT-199, GDC-0199 or venetoclax.

### BRIEF DESCRIPTION OF THE DRAWING

FIG. 1: Antitumor efficacy of therapy with the dual combination of RG6146 and venetoclax, compared to vehicle and mono therapy (Day 14-25).

The term “BET inhibitor” according to the invention refers to agents that prevents activity of BET proteins with an IC₅₀ of about 0.001 μM to about 2 μM.

The term “Bcl-2 inhibitor according to the invention refers to agents that prevents activity of Bcl-2 proteins with an IC₅₀ of about 0.001 μM to about 2 μM

“Salt” refers to salts of the compounds as a pharmaceutically acceptable salt. Such salts can be exemplified by the salts with alkali metals (potassium, sodium, and the like), salts with alkaline-earth metals (calcium, magnesium, and the like), the ammonium salt, salts with pharmaceutically acceptable organic amines (tetramethylammonium, triethylamine, methylamine, dimethylamine, cyclopentylamine, benzylamine, phenethylamine, piperidine, monoethanolamine, diethanolamine, tris(hydroxymethyl)aminomethane, lysine, arginine, N-methyl-D-glucamine, and the like), and acid addition salts (inorganic acid salts (the hydrochloride, hydrobromide, hydroiodide, sulfate, phosphate, nitrate, and the like) and organic acid salts (the acetate, trifluoroacetate, lactate, tartrate, oxalate, fumarate, maleate, benzoate, citrate, methanesulfonate, ethanesulfonate, benzenesulfonate, toluenesulfonate, isethionate, glucuronate, gluconate, and the like)).

“IC₅₀” refers to the concentration of a particular compound required to inhibit 50% of a specific measured activity.

The terms “combination”, “co-administration” or “co-administering” refer to the administration of the BET inhibitor and the Bcl-2 inhibitor according to the invention in one or several formulations. The co-administration can be simultaneous or sequential in either order, wherein preferably there is a time period while both (or all) active agents simultaneously exert their biological activities. The BET inhibitor and the Bcl-2 inhibitor can be co-administered either simultaneously or sequentially. When the therapeutic agents are co-administered sequentially, the dose can for example be administered either on the same day in three separate administrations, or one of the agents can be administered on day 1 and the second and third can be co-administered on day 2 to day 7, preferably on day 2 to 4. Thus in one embodiment the term “sequentially” means within 7 days after the dose of the first component, preferably within 4 days after the dose of the first component; and the term “simultaneously” means at the same time or on the same day. The terms “co-administration” with respect to the maintenance doses of the BET inhibitor and the Bcl-2 inhibitor mean that the maintenance doses can be either co-administered simultaneously, if the treatment cycle is appropriate for both drugs, e.g. every week. Or one of the components (either the Bcl-2 inhibitor or the BET inhibitor) can be administered e.g. every first to third day and the second component can be administered every week. Or the maintenance doses are co-administered sequentially, either within one or within several days.

It is self-evident that the inhibitors are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

The amount of co-administration of the the BET inhibitor and the Bcl-2 inhibitor and the timing of co-administration will depend on the type (species, gender, age, weight, etc.) and condition of the patient being treated and the severity of the disease or condition being treated.

The BET inhibitor is preferably administered subcutaneously.

The BET inhibitor is preferably administered at a dose between about 0.3 mg/kg/d and about 0.65 mg/kg/d.

The BET inhibitor is preferably administered daily for 14 consecutive days every 3 weeks (i.e.. 2 weeks of dosing, 1 week of rest).

The BET inhibitor is preferably administered subcutaneously, at a dose between about 0.3 mg/kg/d and about 0.65 mg/kg/d.

The BET inhibitor is preferably administered subcutaneously, at a dose between about 0.3 mg/kg/d and about 0.65 mg/kg/d for 14 consecutive days every 3 weeks (i.e.. 2 weeks of dosing, 1 week of rest).

The BET inhibitor is preferably RG6146.

The administration of the BET inhibitor, in particular RG6146, can be interrupted for up to 3 weeks, i.e.. 1, 2 or 3 weeks.

The Bcl-2 inhibitor is preferably administered orally.

The Bcl-2 inhibitor is preferably administered at a dose between about 400 mg/d to about 800 mg/d.

The Bcl-2 inhibitor is preferably administered orally, at a dose between about 400 mg/d and about 800 mg/d.

The Bcl-2 inhibitor is preferably administered daily (i.e.. every day). This is called a continuous administration.

The Bcl-2 inhibitor is preferably daily administered orally, at a dose between about 400 mg/d and about 800 mg/d.

The Bcl-2 inhibitor is preferably venetoclax.

The administration cycles of the BET inhibitor and Bcl-2 inhibitor are preferably initiated on the same day.

Depending on the type and severity of the disease, the following amounts can be administered: about 0.3 mg/kg/d to about 0.65 mg/kg/d of the BET inhibitor, preferably RG6146; about 400 mg/d to about 800 mg/d of the Bcl-2 inhibitor, preferably venetoclax.

A particular advantageous combination is about 0.3 mg/kg/d to about 0.65 mg/kg/d of the BET inhibitor, preferably RG6146, every day for 14 consecutive days every 3 weeks (i.e.. 2 weeks of dosing, 1 week of rest); about 400 mg/d to about 800 mg/d continuously (i.e.. every day) of the Bcl-2 inhibitor, preferably venetoclax.

A further particular advantageous combination is about 0.3 mg/kg/d to about 0.65 mg/kg/d of the BET inhibitor, preferably RG6146, subcutaneously every day for 14 consecutive days every 3 weeks (i.e.. 2 weeks of dosing, 1 week of rest); about 400 mg/d to about 800 mg/d continuously (i.e.. every day) and orally of the Bcl-2 inhibitor, preferably venetoclax.

In the above dosing regime, the administration of the BET inhibitor, in particular RG6146, can be interrupted for up to 3 weeks, i.e. 1, 2 or 3 weeks.

In the above dosing regime, the administration of the Bcl-2 inhibitor, in particular venetoclax, can be interrupted for up to 3 weeks, i.e. 1, 2 or 3 weeks.

The recommended dose may vary when there is a further co-administration of a chemotherapeutic agent.

The present invention is useful for preventing or reducing metastasis or further dissemination in such a patient suffering from multiple myeloma. This invention is useful for increasing the duration of survival of such a patient, increasing the progression free survival of such a patient, increasing the duration of response, resulting in a statistically significant and clinically meaningful improvement of the treated patient as measured by the duration of survival, progression free survival, response rate or duration of response. In a preferred embodiment, this invention is useful for increasing the response rate in a group of patients.

In the context of this invention, additional other cytotoxic, chemotherapeutic or anti-cancer agents, or compounds or ionizing radiation that enhance the effects of such agents (e.g. cytokines) may be used. Such molecules are suitably present in combination in amounts that are effective for the purpose intended.

Such additional agents include, for example: alkylating agents or agents with an alkylating action, such as cyclophosphamide (CTX; e.g. cytoxan®), chlorambucil (CHL; e.g. leukeran®), cisplatin (CisP; e.g. platinol®) busulfan (e.g. myleran®), melphalan, carmustine (BCNU), streptozotocin, triethylenemelamine (TEM), mitomycin C, and the like; anti-metabolites, such as methotrexate (MTX), etoposide (VP16; e.g. vepesid®), 6-mercaptopurine (6MP), 6-thiocguanine (6TG), cytarabine (Ara-C), 5-fluorouracil (5-FU), capecitabine (e.g. Xeloda®), dacarbazine (DTIC), and the like; antibiotics, such as actinomycin D, doxorubicin (DXR; e.g. adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. taxol®) and paclitaxel derivatives, the cytostatic agents, glucocorticoids such as dexamethasone (DEX; e.g. decadron®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ethyol®), dactinomycin, mechlorethamine (nitrogen mustard), streptozocin, cyclophosphamide, lomustine (CCNU), doxorubicin lipo (e.g. doxil®), gemcitabine (e.g. gemzar®), daunorubicin lipo (e.g. daunoxome®), procarbazine, mitomycin, docetaxel (e.g. taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine or chlorambucil.

The use of the cytotoxic and anticancer agents described above as well as antiproliferative target-specific anticancer drugs like protein kinase inhibitors in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

In the context of this invention, an effective amount of ionizing radiation may be carried out and/or a radiopharmaceutical may be used. The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, yttrium-90, cesium-137, iridium-192, americium-241, gold-198, cobalt-57, copper-67, technetium-99, iodine-123, iodine-131, and indium-111. Is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in WO 99/60023.

As used herein, a “pharmaceutically acceptable carrier” or “pharmaceutically acceptable excipient” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Pharmaceutical compositions can be obtained by processing the BET inhibitor inhibitor and the Bcl-2 inhibitor according to this invention with pharmaceutically acceptable, inorganic or organic carriers or excipients. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Pharmaceutical compositions of the BET inhibitor inhibitor and the Bcl-2 inhibitor, alone or in combination, can be prepared for storage by mixing an antibody having the desired degree of purity with optional pharmaceutically acceptable carriers, excipients or stabilizers (Remington's Pharmaceutical Sciences 16th edition, Osol, A. (ed.) (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions of the BET inhibitor and of the Bcl-2 inhibitor include those suitable for oral, nasal, topical (including buccal and sublingual), rectal, vaginal and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a Bcl-2 inhibitor or a BET inhibitor which produces a therapeutic effect. Generally, out of one hundred percent, this amount will range from about 1 percent to about 90 percent of active ingredient, preferably from about 5 percent to about 70 percent, most preferably from about 10 percent to about 30 percent. Methods of preparing these compositions include the step of bringing into association a Bcl-2 inhibitor or a BET inhibitor with the carrier and, optionally, one or more accessory ingredients. In general, the pharmaceutical compositions can be prepared by uniformly and intimately bringing into association a Bcl-2 inhibitor and a BET inhibitor with liquid carriers, or finely divided solid carriers, or both, and then, if necessary, shaping the product. Pharmaceutical compositions suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a Bcl-2 inhibitor and a BET inhibitor as an active ingredient. A Bcl-2 inhibitor and a BET inhibitor may also be administered as a bolus, electuary or paste.

In further embodiments of the invention, the BET inhibitor inhibitor and the Bcl-2 inhibitor are formulated into one or two separate pharmaceutical compositions.

The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. (ed.) (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid.

The formulations to be used for in vivo administration must be sterile. This is readily accomplished by filtration through sterile filtration membranes.

The following examples and figures are provided to illustrate the invention and have no limiting character.

### EXAMPLES

### Example 1: In Vivo Antitumor Efficacy

The in vivo antitumor efficacy of BET inhibitor RG6146 in combination with Bcl-2 inhibitor venetoclax (GDC-0199) was evaluated against KMS-12BM MM xenografts.

### Test Agents

BET inhibitor RG6146 was provided as a powder from Roche, Basel, Switzerland and resuspended prior to use. Bcl-2 inhibitor venetoclax was provided by Genentech, South San Francisco, USA and formulated prior to use.

### Cell Line and Culture Conditions

The original KMS-12BM human Multiple Myeloma cell line (MM) was purchased from ATCC (Manassas, V., USA). Expansion of tumor cells for the transplantation was done by the TAP CompacT CellBase Cell Culture Roboter according to the protocol. Tumor cell line was routinely cultured in RPMI 1640 medium, FCS 10% and L-Glutamin 2 mM at 37° C. in a water-saturated atmosphere at 5% CO₂. Culture passage was performed with trypsin/EDTA 1× splitting twice/week and passage 3 used for transplantation.

### Animals

Female CIEA NOG mice (Taconic), age 5-7 weeks at arrival, maintained under specific-pathogen-free condition with daily cycles of 12 h light/12 h darkness according to committed guidelines. Experimental study protocol was reviewed and approved by local government. After arrival animals were maintained in animal facility for one week to get accustomed to new environment and for observation. Continuous health monitoring was carried out on regular basis. Diet food and autoclaved water were provided ad libitum.

### Monitoring

Animals were controlled daily for clinical symptoms and detection of adverse effects. For monitoring throughout the experiment body weight of animals was documented.

### Treatment of Animals

Animal treatment started after randomisation when median tumor size was about 170 mm³. The vehicle was administered ip once daily (QD) on D14-25. BET inhibitor RG6146 ip treatment at 30 mg/kg was done as single agent and in combination on D14-25. Finally, Bcl-2 inhibitor venetoclax was given orally at 100 mg/kg as single agent and in combination on D14-25.

### Antitumor Efficacy

KMS-12BM human MM cells were s.c. inoculated with Matrigel onto female CIEA-NOG mice. Tumor bearing mice were randomized 14 days later to the indicated study groups and compound treatment initiated. Tumor bearing animals were treated with vehicle control, with the BET inhibitor RG6146 at 30 mg/kg or with Bcl-2 inhibitor venetoclax at 100 mg/kg as single agent and in combination thereof. As a result, all compounds given as single agent demonstrated significant anti-tumor efficacy against KMS-12BM xenografts. Briefly, treatment with the BET inhibitor RG6146 resulted in strong significant efficacy with nearly tumor stasis (94% tumor growth inhibition) against KMS-12BM xenografts compared to control. In contrast to this, a weaker activity was noticed after treatment with the Bcl-2 inhibitor venetoclax (49% TGI), whereas superior efficacy was achieved after treatment with the dual combination group including the BET inhibitor RG6164 plus Bcl-2 inhibitor venetoclax.

In more detail the dual combination approach substantially induced tumor regression which reached finally 54%. The strong efficacy of the dual combination arm with tumor regression of KMS-12BM MM xenografts was more than additive compared to the respective single agent arms.

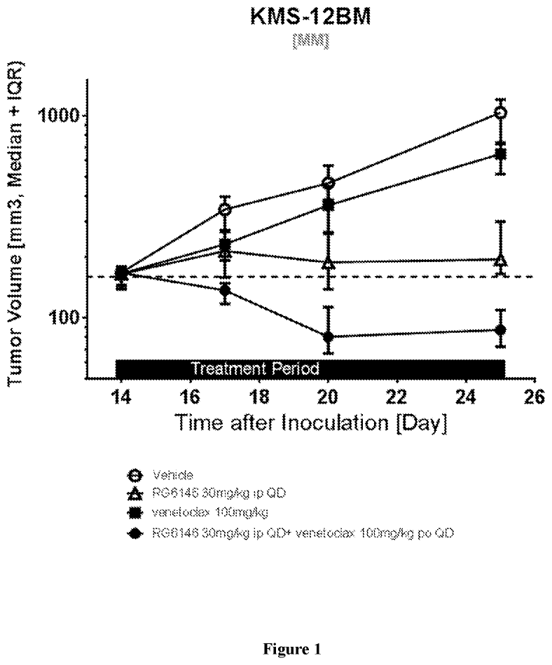

The results are illustrated by Table 1 below and FIG. 1.

TABLE 1
Efficacy of BETi RG6146 and Bcl-2i venetoclax (Day 25)

| | | | | Tumor | |
|---|---|---|---|---|---|
| | Dose | | TGI | Regression | npTCR |
| Compound | (mg/kg) | Schedule | % | (%) | and CI |
| control | — | ip | — | — | — |
| RG6146 | 30 | ip | 94 | 0 | 0.06 |
| | | (QD) | | | (CI 0.03-0.41) |
| venetoclax | 100 | po | 49 | 0 | 0.55 |
| | | (QD) | | | (CI 0.35-0.91) |
| RG6146 + | 30 | ip (QD) | >100 | 54 | −0.09 |
| venetoclax | 100 | po (QD) | | | (CI −0.16-−0.03) |

TCR: Treatment to Control Ratio; pTCR: non-parametric Tumor Control Ratio; CI: Confidence Interval

1-14. (canceled)

15. A method of treating multiple myeloma in a subject in need thereof, comprising administering to said subject a therapeutically effective amount of a BET inhibitor and a Bcl-2 inhibitor.

16. The method of claim 15, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3- (4-methyl-piperazin-1-yl)-propyl]-acetamide.

17. The method of claim 16, wherein the BET inhibitor is administered subcutaneously at a dose between about 0.3 mg/kg/d and about 0.65 mg/kg/d for 14 consecutive days every 3 weeks.

18. The method of claim 15, wherein the Bcl-2 inhibitor is venetoclax.

19. The method of claim 18, wherein the Bcl-2 inhibitor is daily administered orally at a dose between about 400 mg/d and about 800 mg/d

20. The method of claim 15, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3- (4-methyl-piperazin-1-yl)-propyl]-acetamide, and the Bcl-2 inhibitor is venetoclax.

21. The method of claim 15, wherein the BET inhibitor is co-administered with the Bcl-2 inhibitor.

22. The method of claim 15, wherein each of said BET inhibitor and said Bcl-2 inhibitor are administered separately.

23. The method of claim 15, said method further comprising administering a therapeutically effective amount of one or more additional other cytotoxic, chemotherapeutic or anti-cancer agents.

24. A pharmaceutical composition comprising a BET inhibitor, a Bcl-2 inhibitor and one or more pharmaceutically acceptable excipients.

25. The pharmaceutical composition of claim 24, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide.

26. The pharmaceutical composition of claim 24, wherein the Bcl-2 inhibitor is venetoclax.

27. The pharmaceutical composition of claim 24, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide and the Bcl-2 inhibitor is venetoclax.

28. The pharmaceutical composition of claim 24, said composition further comprising one or more additional other cytotoxic, chemotherapeutic or anti-cancer agents.

29. A kit comprising a BET inhibitor and a Bcl-2 inhibitor for the simultaneous, separate or sequential administration of said BET inhibitor and Bcl-2 inhibitor.

30. The kit of claim 29, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide.

31. The kit of claim 29, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide, and the Bcl-2 inhibitor is venetoclax.

32. The kit of claim 29, said kit further comprising one or more additional other cytotoxic, chemotherapeutic or anti-cancer agents.
